(19) United States
(12) Patent Application Publication
(10) Pub. No.: US 2024/0081716 A1
(43) Pub. Date: Mar. 14, 2024

(54) HEART SIGNAL MONITORING METHOD AND APPARATUS, STORAGE MEDIUM AND ELECTRONIC DEVICE

(71) Applicant: United InnoMed (Shanghai) Limited, Shanghai (CN)

(72) Inventor: Li WANG, Shanghai (CN)

(73) Assignee: United InnoMed (Shanghai) Limited, Shanghai (CN)

(21) Appl. No.: 18/518,799

(22) Filed: Nov. 24, 2023

Related U.S. Application Data
(63) Continuation of application No. PCT/CN2022/093863, filed on May 19, 2022.

(30) Foreign Application Priority Data
May 28, 2021 (CN) 202110594940.8

(51) Int. Cl. A61B 5/352; A61B 5/355

(52) U.S. Cl. CPC A61B 5/352; A61B 5/355

(57) ABSTRACT

Disclosed are a heart signal monitoring method, a heart signal monitoring apparatus, a storage medium and an electronic device, relating to the field of medical data processing technologies. The heart signal monitoring method includes: determining, based on a first monitoring wave in an electrocardiogram, first threshold value information corresponding to the first monitoring wave within a preset monitoring time interval; and determining, based on the first threshold value information, first monitoring assessment information corresponding to the first monitoring wave. The present disclosure achieves accurate monitoring of heart signals. Specifically, the preset monitoring time interval for the first monitoring wave is limited based on the actual situation of the first monitoring wave, and the first monitoring assessment information is determined based on the first threshold value information, such that the actual situation of the first monitoring wave is taken into full consideration, thereby improving precision of monitoring of a heart signal.

### CROSS-REFERENCE TO RELATED APPLICATIONS

This application is a continuation of International Application No. PCT/CN2022/093863, filed on May 19, 2022, which claims priority to Chinese Patent Application No. 202110594940.8, filed on May 28, 2021. The disclosures of the aforementioned applications are hereby incorporated by reference in their entireties.

### TECHNICAL FIELD

The present disclosure relates to the field of medical data processing technologies, and in particular, to a heart signal monitoring method, a heart signal monitoring apparatus, a storage medium and an electronic device.

### BACKGROUND

As is well known, it is self-evident that a heart is of great importance as an organ that provides power for blood flow. Effective monitoring of heart signals not only helps timely detection of physical abnormalities in patients, which is conducive to diagnosis and treatment of doctors, but also improves accuracy of medical devices such as heart rhythm management devices (for example, an implantable cardioverter-defibrillator, a cardiac resynchronization treatment device, a pacemaker, and so on) to assist in improving curative effect, as well as comfort and safety of patients.

However, according to existing heart signal monitoring methods, various actual electric activities of a heart and their variation along with a condition of illness may not be fully considered and/or responded to. Therefore, the accuracy of the existing method needs to be improved, for example, by preventing and reducing over-sensing or under-sensing.

### SUMMARY

The present disclosure is provided to address technical issues mentioned above. Embodiments of the present disclosure provide a heart signal monitoring method and apparatus, a storage medium and an electronic device.

In an aspect, an embodiment of the present disclosure provides a heart signal monitoring method, including: determining, based on a first monitoring wave in an electrocardiogram, first threshold value information corresponding to the first monitoring wave within a preset monitoring time interval, where the first threshold value information is used for monitoring the first monitoring wave; and determining, based on the first threshold value information, first monitoring assessment information corresponding to the first monitoring wave, where the first monitoring assessment information includes whether the first monitoring wave being nearly to be sensed.

In an embodiment of the present disclosure, the determining, based on the first threshold value information, first monitoring assessment information corresponding to the first monitoring wave includes: determining, based on second monitoring waves in the electrocardiogram corresponding to the first monitoring wave, a plurality of first periods corresponding to the first monitoring wave; determining peak information of the first monitoring wave corresponding to each of the plurality of first periods; and determining, based on the peak information of the first monitoring wave corresponding to each of the plurality of first periods and the first threshold value information, the first monitoring assessment information; where the first monitoring wave is a T wave, and the second monitoring wave is an R wave; and/or the first monitoring wave is a far-field R (FFR) wave, and the second monitoring wave is a P wave.

In an embodiment of the present disclosure, the determining, based on the peak information of the first monitoring wave corresponding to each of the plurality of first periods and the first threshold value information, the first monitoring assessment information includes: determining, based on the peak information of the first monitoring wave corresponding to each of the plurality of first periods and the first threshold value information, first region label information corresponding to each of the plurality of first periods, where the first region label information includes adjustment label information, focus label information, and maintaining label information; and determining, based on the first region label information corresponding to each of the plurality of first periods, the first monitoring assessment information.

In an embodiment of the present disclosure, the determining, based on the peak information of the first monitoring wave corresponding to each of the plurality of first periods and the first threshold value information, first region label information of the first period corresponding to each of the plurality of first periods includes: performing calculation to obtain first difference value information according to the peak information of the first monitoring wave corresponding to each of the plurality of first periods and the first threshold value information; performing calculation to obtain first ratio information according to the first difference value information and the first threshold value information; and determining, based on the first ratio information, the first region label information.

In an embodiment of the present disclosure, the determining, based on the first ratio information, the first region label information includes: when the first ratio information is less than a first preset value, determining the first region label information to be the adjustment label information, where the first preset value is greater than or equal to 5%, and less than or equal to 15%; and/or when the first ratio information is greater than or equal to the first preset value and less than a second preset value, determining the first region label information to be the focus label information, where the second preset value is greater than or equal to 15%, and less than or equal to 30%; the determining, based on the first region label information corresponding to each of the plurality of first periods, the first monitoring assessment information includes: when the first region label information corresponding to each of a plurality of consecutive first periods is the adjustment label information, determining that the first monitoring wave is nearly to be sensed, where the plurality of consecutive first periods include two or more than two first periods.

In an embodiment of the present disclosure, the determining, based on second monitoring waves in the electrocardiogram corresponding to the first monitoring wave, a plurality of first periods corresponding to the first monitoring wave includes: determining occurrence period information corresponding to the second monitoring waves; determining a plurality of first monitoring nodes of the second monitoring waves; determining a node after a preset period starting from each of the plurality of first monitoring nodes as a start time node of the first period corresponding to the first monitoring node, where the plurality of first periods and the plurality of first monitoring nodes have a one-to-one correspondence; determining, based on a preset percentage of the occurrence period information, an end time node of the first period corresponding to the first monitoring node; and determining, based on the start time node and the end time node, the first period corresponding to the first monitoring node.

In an embodiment of the present disclosure, the first monitoring wave is a T wave in an electrocardiogram, and the second monitoring wave is an R wave in an electrocardiogram; and/or, the first monitoring wave is an FFR wave of electrocardiogram signals that primarily involve atrial electrical activity (such as atrial electrocardiogram), and the second monitoring wave is a P wave in an electrocardiogram.

In another aspect, an embodiment of the present disclosure provides a heart signal monitoring method, including: determining, based on a second monitoring wave in an electrocardiogram corresponding to a first monitoring wave, a threshold node and second threshold value information corresponding to the second monitoring wave within a preset monitoring time interval, where the threshold node and the second threshold value information are used for sensing the second monitoring wave, and the second threshold value information is used for voiding sensing the first monitoring wave; and determining, based on the threshold node and the second threshold value information, second monitoring assessment information corresponding to the second monitoring wave, where the second monitoring assessment information includes whether the first monitoring wave being nearly not to be sensed.

In an embodiment of the present disclosure, the determining, based on the threshold node and the second threshold value information, second monitoring assessment information corresponding to the second monitoring wave includes: determining, based on the threshold node and the second threshold value information, a plurality of second periods corresponding to the second monitoring wave, where the second threshold value information is linear or exponential decay information; determining start point value information and peak information corresponding to each of the plurality of second periods; and determining, based on the start point value information and the peak information corresponding to each of the plurality of second periods, the second monitoring assessment information.

In an embodiment of the present disclosure, the determining, based on the start point value information and the peak information corresponding to each of the plurality of second periods, the second monitoring assessment information includes: determining, based on the start point value information and the peak information corresponding to each of the plurality of second periods, second region label information corresponding to each of the plurality of second periods, where the second region label information includes adjustment label information, focus label information, and maintaining label information; and determining, based on the second region label information corresponding to each of the plurality of second periods, the second monitoring assessment information.

In an embodiment of the present disclosure, the determining, based on the start point value information and the peak information corresponding to each of the plurality of second periods, second region label information corresponding to each of the plurality of second periods includes: performing calculation to obtain second difference value information according to the peak information and the start point value information; performing calculation to obtain second ratio information according to the second difference value information and the peak information; and determining, based on the second ratio information, the second region label information.

In an embodiment of the present disclosure, the determining, based on the second ratio information, the second region label information includes: when the second ratio information is less than a third preset value, determining the second region label information to be the adjustment label information, where the third preset value is greater than or equal to 5%, and less than or equal to 15%; and/or when the second ratio information is greater than or equal to the third preset value and less than a fourth preset value, determining the second region label information to be the focus label information, where the fourth preset value is greater than or equal to 15%, and less than or equal to 30%;

the determining the second monitoring assessment information based on the second region label information corresponding to each of the plurality of second periods includes: when the second region label information corresponding to each of a plurality of consecutive first periods is the adjustment label information, determining that the second monitoring wave is nearly not to be sensed, where the plurality of consecutive second periods include two or more than two second periods.

In an embodiment of the present disclosure, the determining, based on the threshold node and the second threshold value information, a plurality of second periods corresponding to the second monitoring wave includes: determining, based on the threshold nodes, a plurality of second monitoring nodes of the second monitoring wave; and determining a preset period after each of the plurality of second monitoring nodes as the second period corresponding to the second monitoring node.

In an embodiment of the present disclosure, the heart signal monitoring method provided by the present disclosure further includes: determining, based on the first monitoring wave in the electrocardiogram, first threshold value information corresponding to the first monitoring wave within the preset monitoring time interval, where the first threshold value information is used for monitoring the first monitoring wave; and determining, based on the first threshold value information, first monitoring assessment information corresponding to the first monitoring wave, where the first monitoring assessment information includes whether the first monitoring wave being nearly to be sensed.

In an embodiment of the present disclosure, the heart signal monitoring method provided by the present disclosure further includes: when the first monitoring assessment information is that the first monitoring wave is nearly to be sensed, and/or the second monitoring assessment information is that the second monitoring wave is nearly not to be sensed, adjusting, based on the first monitoring assessment information and/or the second monitoring assessment information, parameters of a sensing function of a monitoring device; and/or sending alarm information based on the first monitoring assessment information and/or the second monitoring assessment information; and/or adjusting, based on the first monitoring assessment information and/or the second monitoring assessment information, at least one of the first threshold value information, the threshold node, the second threshold value information, and a highest sensitivity threshold; and/or storing relevant data corresponding to the first monitoring assessment information and/or the second monitoring assessment information for information post-processing and evaluating by a doctor.

In an embodiment of the present disclosure, the monitoring device includes an implantable medical device, and the implantable medical device includes a pacemaker.

In an embodiment of the present disclosure, the adjusting, based on the first monitoring assessment information and/or the second monitoring assessment information, at least one of the first threshold value information, the threshold node, the second threshold value information, and a highest sensitivity threshold information includes: adjusting, based on the first monitoring assessment information and/or the second monitoring assessment information, a sensing parameter of a sensing amplifier, to adjust at least one of the first threshold value information, the threshold node, the second threshold value information, and the highest sensitivity threshold.

In another aspect, a non-transitory computer-readable storage medium is provided by an embodiment of the present disclosure, where a computer program is stored for performing the heart signal monitoring method according to the embodiments mentioned above.

In another aspect, an embodiment of the present disclosure provides a medical electronic device, including: a processor; and a memory, configured to store executable instructions of the processor, where the processor is used for implementing the heart signal monitoring method according to the embodiments mentioned above.

According to the heart signal monitoring method provided by the embodiments of the present disclosure, accurate monitoring of heart signals is realized by determining, based on a first monitoring wave, first threshold value information corresponding to the first monitoring wave within a preset monitoring time interval, and then determining, based on the first threshold value information, first monitoring assessment information corresponding to the first monitoring wave. Specifically, according to the embodiment of the present disclosure, the preset monitoring time interval for the first monitoring wave is limited based on an actual situation of the first monitoring wave, and the first monitoring assessment information corresponding to the first monitoring wave is determined based on the first threshold value information corresponding to the first monitoring wave. It can be seen that the actual situation of the first monitoring wave is fully considered according to the embodiment of the present disclosure, so that the monitoring accuracy of heart signals is improved.

### BRIEF DESCRIPTION OF THE DRAWINGS

To make the above and other purposes, features, and advantages of the present disclosure more apparent, a more detailed description of embodiments of the present disclosure will be given with reference to the accompanying drawings. The accompanying drawings are used to provide a further understanding of the embodiments of the present disclosure, and form a part of the specification which explains the disclosure with the embodiments and do not constitute a limitation on the disclosure. In the accompanying drawings, the same numeral usually represents the same component or step.

FIG. 1 is a schematic diagram of an applicable scenario of an embodiment of the present disclosure.

FIG. 2 is another schematic diagram of an applicable scenario of an embodiment of the present disclosure.

FIG. 3 is a schematic flowchart of a heart signal monitoring method according to an embodiment of the present disclosure.

FIG. 4 is a schematic flowchart of determining, based on first threshold value information, first monitoring assessment information corresponding to a first monitoring wave according to an embodiment of the present disclosure.

FIG. 5 is a schematic flowchart of determining, based on peak information of a first monitoring wave corresponding to each of a plurality of first periods and first threshold value information, first monitoring assessment information according to an embodiment of the present disclosure.

FIG. 6 is a schematic flowchart of determining, based on peak information of a first monitoring wave corresponding to each of a plurality of first periods and first threshold value information, first region label information corresponding to each of the plurality of first periods according to an embodiment of the present disclosure.

FIG. 7 is a schematic flowchart of determining, based on a second monitoring wave corresponding to a first monitoring wave, a plurality of first periods corresponding to the first monitoring wave according to an embodiment of the present disclosure.

FIG. 8 is a schematic diagram of a waveform during a paced rhythm monitoring time interval according to an embodiment of the present disclosure.

FIG. 9 is another schematic flowchart of a heart signal monitoring method according to an embodiment of the present disclosure.

FIG. 10 is a schematic flowchart of determining, based on a threshold node and second threshold value information, second monitoring assessment information corresponding to a second monitoring wave according to an embodiment of the present disclosure.

FIG. 11 is a schematic flowchart of determining, based on start point value information and peak information corresponding to each of a plurality of second periods, second monitoring assessment information according to an embodiment of the present disclosure.

FIG. 12 is a schematic flowchart of determining, based on start point value information and peak information corresponding to the second period, second region label information of second period according to an embodiment of the present disclosure.

FIG. 13 is a schematic flowchart of determining, based on a threshold node and second threshold value information, a plurality of second periods corresponding to a second monitoring wave according to an embodiment of the present disclosure.

FIG. 14 is a schematic diagram of a waveform during an intrinsic rhythm monitoring time interval according to an embodiment of the present disclosure.

FIG. 15 is still another schematic flowchart of a heart signal monitoring method according to an embodiment of the present disclosure.

FIG. 16 is a schematic structural diagram of a heart signal monitoring apparatus according to an embodiment of the present disclosure.

FIG. 17 is a schematic structural diagram of a second determination module according to an embodiment of the present disclosure.

FIG. 18 is a schematic structural diagram of a first monitoring assessment information determination unit according to an embodiment of the present disclosure.

FIG. 19 is a schematic structural diagram of a first period determination unit according to an embodiment of the present disclosure.

FIG. 20 is another schematic structural diagram of a heart signal monitoring apparatus according to an embodiment of the present disclosure.

FIG. 21 is a schematic structural diagram of a fourth determination module according to an embodiment of the present disclosure.

FIG. 22 is a schematic structural diagram of a second monitoring assessment information determination unit according to an embodiment of the present disclosure.

FIG. 23 is a schematic structural diagram of a second period determination unit according to an embodiment of the present disclosure.

FIG. 24 is still another schematic structural diagram of a heart signal monitoring apparatus according to an embodiment of the present disclosure.

FIG. 25 is a schematic structural diagram of an electronic device according to an embodiment of the present disclosure.

### DETAILED DESCRIPTION OF THE EMBODIMENTS

A detailed description of exemplary embodiments will be given below with reference to the accompanying drawings. Apparently, the described embodiments are only a part, but not all of the embodiments of the present disclosure. It should be understood that the present disclosure is not limited to the exemplary embodiments described herein.

### Overview

A heart signal, such as an R wave, a T wave, a P wave, and a far-field R (FFR) wave, refers to an electric signal that characterizes heart activities. The R wave, also known as QRS wave complex, characterizes time and potential changes during depolarization of left and right ventricular muscles. The T wave characterizes potential changes generated by rapid repolarization of ventricular muscles. The P wave characterizes potential changes generated by atrial depolarization, and reflects a depolarization process of a left atria and a right atria. The FFR wave characterizes depolarization electric activities of ventricular muscles sensed by an electrode, such as an atrial electrode, located outside the ventricle.

Currently, a medical device, such as an implantable heart pacemaker and an implantable cardioverter-defibrillator (ICD), usually achieves heart signal monitoring through a fixed sensing threshold algorithm or a sensing threshold algorithm automatically adjusted based on amplitudes of the R wave or the P wave. However, monitoring accuracy of the medical device mentioned above still needs improvement in some clinical situations. Especially for situations that the R wave or the P wave significantly decreases, and/or the T wave significantly increases with time and/or changes along with a myocardial condition of a patient, it is difficult for existing sensing threshold algorithms to accurately monitor the electric activities of the heart, which may result in under-sensing of the R wave or over-sensing of the T wave, thereby affecting accuracy of arrhythmic diagnosis through the medical device. Although schemes for changing a sensing threshold do exist in the prior art, situations such as under-sensing of the R wave, over-sensing of the T wave, and/or under-sensing of ventricular fibrillation (VF) may occur, which may result in false positive or false negative result for arrhythmia and lead to improper treatment. When similar situations occur in clinical practice, doctors need to solve them through ICD program control (non-invasive) or even re-placement of relevant electrodes (invasive). For example, if an R wave of a patient significantly decreases, the doctor may need to reduce or eliminate under-sensing of the R wave by increasing sensitivity of a sensing amplifier; if a T wave is sensed because of over-sensing, the doctor may need to reduce the sensitivity of the sensing amplifier. However, it often fails to achieve good results. Meanwhile, these problems are often discovered after a patient is in a bad situation. For example, failure to detect the occurrence of VF may cause the patient to faint, or physiological tachycardia caused by exercise is detected as VT/VF, resulting in improper discharge that causes patient pain, and those problems are discovered and treated only after the patient is send to the hospital, instead of informing or automatically adjusting in advance to reduce or avoid similar events.

Furthermore, in recent years, with rapid development of intelligent medical technologies, implantation surgeries based on medical devices such as pacemakers or ICDs have become increasingly popular. More hospitals are implementing such therapies, and more doctors are performing related surgeries. However, currently, doctors have relatively limited experience in implantation and management of implanted devices, especially in small and remote hospitals. Therefore, how to improve the accuracy of medical devices for monitoring heart signals and rhythms and reduce misjudgments to better assist doctors in diagnosis and treatment and to simultaneously increase patient satisfaction and comfort with the treatment has become an urgent problem to be solved. And it is more worthy of attention and specific solutions under the circumstance that the relationship between patients and doctors is relatively tense.

Based on the above technical issues, a basic concept of the present disclosure is to provide a heart signal monitoring method and apparatus, a storage medium and an electronic device.

A heart signal monitoring method provided by an embodiment of the present disclosure includes: determining, based on a first monitoring wave in an electrocardiogram, first threshold value information corresponding to the first monitoring wave within a preset monitoring time interval, where the first threshold value information is used for monitoring the first monitoring wave; and determining, based on the first threshold value information, first monitoring assessment information corresponding to the first monitoring wave.

According to the heart signal monitoring method provided by the embodiments of the present disclosure, accurate monitoring of heart signals is realized by determining, based on a first monitoring wave, first threshold value information corresponding to the first monitoring wave within a preset monitoring time interval, and then determining, based on the first threshold value information, first monitoring assessment information corresponding to the first monitoring wave. Specifically, according to the embodiment of the present disclosure, the preset monitoring time interval for the first monitoring wave is limited based on an actual situation of the first monitoring wave, and the first monitoring assessment information corresponding to the first monitoring wave is determined based on the first threshold value information corresponding to the first monitoring wave. It can be seen that the actual situation of the first monitoring wave is fully considered according to the embodiment of the present disclosure, so that the monitoring accuracy of heart signals is improved.

After introduction of the basic concept of the present disclosure, various non-limiting embodiments of the present disclosure will be provided below with reference to the accompanying drawings.

### Exemplary Scenario

FIG. 1 is a schematic diagram of an applicable scenario of an embodiment of the present disclosure. As shown in FIG. 1, the applicable scenario of the embodiment of the present disclosure includes a server 1 and a medical device 2. There is a communication connection relationship between the server 1 and the medical device 2.

Specifically, the medical device 2 is used to collect a first monitoring wave in an electrocardiogram corresponding to a subject. Exemplarily, the first monitoring wave is a T wave or an FFR wave. The server 1 is used to determine, based on the first monitoring wave, first threshold value information corresponding to the first monitoring wave within a preset monitoring time interval, where the first threshold value information is used for monitoring the first monitoring wave; and determine, based on the first threshold value information, first monitoring assessment information corresponding to the first monitoring wave. That is, a heart signal monitoring method is implemented in this scenario.

The heart signal monitoring method is implemented by using the server 1 in the above scenario shown in FIG. 1, so that adaptability to the scenario may be improved, and a computational amount and storage capacity of the medical device 2 may be effectively reduced.

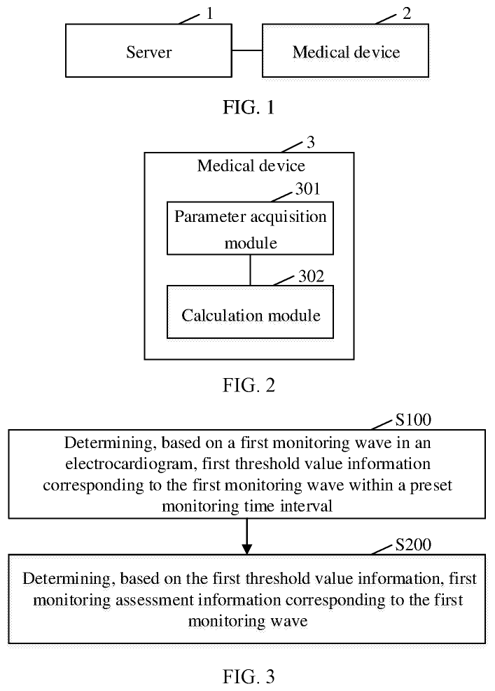

It should be noted that the present disclosure may also be applied to another scenario. FIG. 2 is another schematic diagram of an applicable scenario of an embodiment of the present disclosure. Specifically, this scenario includes a medical device 3, and the medical device 3 includes a parameter acquisition module 301 and a calculation module 302.

Specifically, the parameter acquisition module 301 in the medical device 3 is used to collect a first monitoring wave in an electrocardiogram corresponding to a subject. The calculation module 302 is used to determine, based on the first monitoring wave, first threshold value information corresponding to the first monitoring wave within a preset monitoring time interval, where the first threshold value information is used for monitoring the first monitoring wave; and determine, based on the first threshold value information, first monitoring assessment information corresponding to the first monitoring wave. That is, a heart signal monitoring method is implemented in this scenario.

The heart signal monitoring method is implemented through the medical device 3 in the above scenario shown in FIG. 2, so that there is no need for data transmission operations with a related device such as a server. Therefore, real-time of the heart signal monitoring method may be ensured in the above scenario.

Exemplarily, in the scenarios shown in FIG. 1 and FIG. 2, a communication module and a feedback module may be also included. For example, in FIG. 1, there is a communication connection relationship between the communication module and the medical device 2, and the feedback module has a communication connection relationship with the server 1 and the medical device 2 respectively. For another example, in FIG. 2, the calculation module 302 has a communication connection relationship with the communication module and the feedback module respectively. Specifically, the communication module is used to send information (such as warning information) to concerned person (such as doctors, patients themselves, and patients' families), and the feedback module is used to modify, based on a calculation result of the server 1, a parameter of a relevant sensing module in the medical device 2.

It should be noted that the medical device mentioned in the above scenario may be either an implantable medical device (IMD), such as a pacemaker, an Implantable Cardioverter-Defibrillator (ICD), a Cardiac Resynchronization Treatment Defibrillator (CRT-D), or a subQ ICDs, and may also be a non-implantable medical device, such as a Wearable Implantable Cardioverter-Defibrillator (WCD), an extracorporeal defibrillator, such as an automated external defibrillator (AED) used in hospitals, and so on. There is no unified limitation on this in the embodiments of the present disclosure.

### Exemplary Method

FIG. 3 is a schematic flowchart of a heart signal monitoring method according to an embodiment of the present disclosure. As shown in FIG. 3, the heart signal monitoring method provided by this embodiment includes the following steps.

Step S100: determining, based on a first monitoring wave in an electrocardiogram, first threshold value information corresponding to the first monitoring wave within a preset monitoring time interval.

Exemplarily, the first threshold value information is used to monitor the first monitoring wave. For example, the first monitoring wave is a T wave, and the first threshold value information is a fixed threshold value. For another example, the first monitoring wave is a T wave, and the first threshold value information is a linear decay threshold value.

In a practical application process, if the T wave is very close to the first threshold value information, a false sensing of the T wave may be generated, so that relevant parameters (such as an amplification factor) of the monitoring device may need to be adjusted, or a doctor may need to be informed. In an embodiment of the present disclosure, the first threshold value information includes decay threshold value information of the first monitoring wave, such as decay threshold value information when peak information of the first monitoring wave is measured.

Exemplarily, the preset monitoring time interval includes a paced rhythm monitoring time interval and an intrinsic rhythms monitoring time interval. Therein, the paced rhythm monitoring time interval refers to a monitoring time interval of heart beat caused by a medical device such as a pacemaker for auxiliary treatment; the intrinsic rhythms monitoring time interval refers to a monitoring time interval of a subject's own heart rate without any medical device such as the pacemaker for auxiliary treatment.

In an embodiment of the present disclosure, the preset monitoring time interval is a paced rhythm monitoring time interval of a ventricle and the first monitoring wave includes the T wave. That is, a ventricular signal of the heart is monitored during the paced rhythm monitoring time interval. In another embodiment of the present disclosure, the preset monitoring time interval is a paced rhythm monitoring time interval of an atrium, and the first monitoring wave includes the FFR wave. That is, an atrial signal of the heart is monitored during the paced rhythm monitoring time interval of the atrium.

Step S200: determining, based on the first threshold value information, first monitoring assessment information corresponding to the first monitoring wave.

Exemplarily, the first monitoring assessment information includes whether it is possible for the first monitoring wave nearly to be sensed, that is, an over-sensing (alternatively it can be taken as an assessment of possibility of over-sensing of the first monitoring wave), so that the relevant sensing parameters of the implanted medical device may be adjusted based on the first monitoring assessment information, and/or doctors may be assisted in diagnosis and treatment based on the first monitoring assessment information.

According to the heart signal monitoring method provided by the embodiment of the present disclosure, accurate monitoring of heart signals may be realized by determining, based on the first monitoring wave in the electrocardiogram, first threshold value information corresponding to the first monitoring wave within a preset monitoring time interval, and then determining, based on the first threshold value information, first monitoring assessment information corresponding to the first monitoring wave. Specifically, according to the embodiment of the present disclosure, the preset monitoring time interval for the first monitoring wave is limited based on an actual situation of the first monitoring wave, and the first monitoring assessment information corresponding to the first monitoring wave is determined based on the first threshold value information corresponding to the first monitoring wave. It can be seen that the actual situation of the first monitoring wave is fully considered according to the embodiment of the present disclosure, so that monitoring accuracy of the heart signals is improved.

FIG. 4 is a schematic flowchart of determining, based on first threshold value information, first monitoring assessment information corresponding to a first monitoring wave according to an embodiment of the present disclosure. The embodiment shown in FIG. 4 is an extension on the basis of the embodiment shown in FIG. 3. A difference between the embodiments shown in FIG. 4 and FIG. 3 will be described in the following, and similarities will not be repeated herein.

As shown in FIG. 4, in an embodiment of the present disclosure, the determining the determining, based on the first threshold value information, first monitoring assessment information corresponding to the first monitoring wave includes the following steps.

Step S210: determining, based on a second monitoring waves in the electrocardiogram corresponding to the first monitoring wave, a plurality of first periods corresponding to the first monitoring wave.

It should be understood that the plurality of first periods mentioned in step S210 belong to the preset monitoring time interval.

Exemplarily, if the first monitoring wave is a T wave, then the second monitoring wave corresponding to the first monitoring wave is an R wave. That is, monitoring on an electric activity of a ventricle of heart may be realized based on the first monitoring wave and the second monitoring wave. For another example, if the first monitoring wave is an FFR wave, then the second monitoring wave corresponding to the first monitoring wave is a P wave. That is, monitoring on an electric activity of an atria of heart may be realized based on the first monitoring wave and the second monitoring wave.

Step S220: determining peak information of the first monitoring wave corresponding to each of the plurality of first periods.

Exemplarily, the peak information of the first monitoring wave corresponding to the first period refers to peak information of the first monitoring wave sensed during the first period.

Step S230: determining, based on the peak information of the first monitoring wave corresponding to each of the plurality of first periods and the first threshold value information, the first monitoring assessment information.

Exemplarily, based on the peak information of the first monitoring wave corresponding to each of the plurality of first periods and the first threshold value information, label information corresponding to each of the plurality of first periods (namely, first region label information mentioned in the following embodiment) is determined, then the first monitoring assessment information is determined based on the label information corresponding to each of the plurality of the first periods.

According to the heart signal monitoring method provided by the embodiment of the present disclosure, a purpose of determining the first monitoring assessment information corresponding to the first monitoring wave on the basis of the first threshold value information is realized by determining, based on second monitoring waves in the electrocardiogram corresponding to the first monitoring wave, a plurality of first periods corresponding to the first monitoring wave; determining peak information of the first monitoring wave corresponding to each of the plurality of first periods; and determining, based on the peak information of the first monitoring wave corresponding to each of the plurality of first periods and the first threshold value information, the first monitoring assessment information. In this embodiment, as the preset monitoring time interval is divided into the plurality of first periods, it is possible to periodically evaluate a monitoring status of heart signals, so that the first monitoring assessment information with a higher accuracy may be obtained. In addition, if the first threshold value information includes the decay threshold value information of the first monitoring wave, a monitoring situation (or avoiding monitoring) of the first monitoring wave may be more accurately and comprehensively evaluated by using the peak information corresponding to each of the plurality of first periods and the first threshold value information of the first monitoring wave, thereby obtaining the first monitoring assessment information with more reference significance and a higher accuracy.

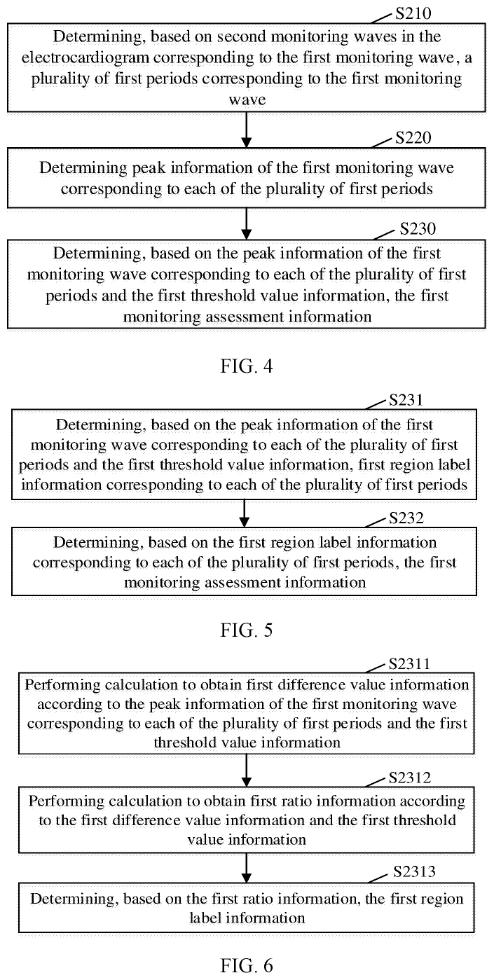

FIG. 5 is a schematic flowchart of determining, based on peak information of a first monitoring wave corresponding to each of a plurality of first periods and first threshold value information, first monitoring assessment information according to an embodiment of the present disclosure. The embodiment shown in FIG. 5 is an extension on the basis of the embodiment shown in FIG. 4. A difference between the embodiments shown in FIG. 5 and FIG. 4 will be described in the following, and similarities will not be repeated herein.

As shown in FIG. 5, in an embodiment of the present disclosure, the determining, based on peak information of a first monitoring wave corresponding to each of a plurality of first periods and first threshold value information, first monitoring assessment information includes the following steps.

Step S231: determining, based on the peak information of the first monitoring wave corresponding to each of the plurality of first periods and the first threshold value information, first region label information of the first period corresponding to each of the plurality of first periods.

Exemplarily, the first region label includes an adjustment label, a focus label, and a maintaining label. Correspondingly, the first region label information includes adjustment label information, focus label information, and maintaining label information. The adjustment label represents that a sensing parameter (such as the first threshold value information mentioned above) corresponding to the heart signal need to be adjusted. The focus label represents that there is an abnormality in the sensing parameter corresponding to the heart signal, which requires special attention but no adjustment temporarily. The maintaining label represents that the sensing parameter corresponding to the heart signal is appropriate and do not need to be adjusted.

Step S232: determining, based on the first region label information corresponding to each of the plurality of first periods, the first monitoring assessment information.

For example, if the first region label information corresponding to each of six consecutive first periods mentioned in step S232 is the adjustment label information, then it indicates that the sensing parameter corresponding to the heart signal need to be adjusted.

According to the heart signal monitoring method provided by the embodiment of the present disclosure, a purpose of determining the first monitoring assessment information on the basis of the peak information of the first monitoring wave corresponding to each of the plurality of first periods and the first threshold value information is realized by determining, based on the peak information of the first monitoring wave corresponding to each of the plurality of first periods and the first threshold value information, first region label information corresponding to each of the plurality of first periods; and determining, based on the first region label information corresponding to each of the plurality of first periods, the first monitoring assessment information. As heart signals of both patients with and without heart disease are very complex, rationality and accuracy of the determined first monitoring assessment information may be improved by monitoring in different periods and determining the first monitoring assessment information in combination with the first region label information corresponding to each period.

FIG. 6 is a schematic flowchart of determining, based on peak information of a first monitoring wave corresponding to each of a plurality of first periods and first threshold value information, first region label information corresponding to each of the plurality of first periods according to an embodiment of the present disclosure. The embodiment shown in FIG. 6 is an extension on the basis of the embodiment shown in FIG. 5. A difference between the embodiments shown in FIG. 6 and FIG. 5 will be described in the following, and similarities will not be repeated herein.

As shown in FIG. 6, in an embodiment of the present disclosure, the determining, based on peak information of a first monitoring wave corresponding to each of a plurality of first periods and first threshold value information, first region label information corresponding to each of the plurality of first periods includes the following steps.

Step S2311: performing calculation to obtain first difference value information according to the peak information of the first monitoring wave corresponding to the first period and the first threshold value information.

Exemplarily, the first monitoring wave is a T wave, the peak information of the first monitoring wave is represented by Tₚₙ, and the first threshold value information is decay threshold value information corresponding to a time point when Tₚₙ is sensed, represented by Tₜₙ. Then, the first difference value information T₁ₙ mentioned in step S2311 may be represented by the following equation (1).

T₁ₙ=Tₜₙ−Tₚₙ (1)

It should be understood that in this embodiment, n represents different first periods and is a positive integer greater than or equal to 1.

Step S2312: performing calculation to obtain first ratio information according to the first difference value information and the first threshold value information.

Following the above notations, the first ratio information Ten may be represented by the following equation (2).

$${T}_{2 n} = \frac{{T}_{1 n}}{{T}_{tn}} = \frac{{T}_{tn} - {T}_{pn}}{{T}_{tn}} \qquad ( 2 )$$

Step S2313: determining, based on the first ratio information, the first region label information.

Specifically, when the first ratio information is less than a first preset value, the first region label information is determined to be the adjustment label information. And the first preset value is greater than or equal to 5%, and less than or equal to 15%. When the first ratio information is greater than or equal to the first preset value and less than a second preset value, the first region label information is determined to be the focus label information, and the second preset value is greater than or equal to 15%, and less than or equal to 30%. When the first ratio information is greater than or equal to the second preset value, the first region label information is determined to be the maintaining label information.

For example, if the first monitoring wave is a T wave and the first ratio information Ten is less than 10%, the first region label information is determined to be “potential over-sensing of T wave”, which is also known as the adjustment label information. Then, correspondingly, if the first region label information corresponding to each of six consecutive first periods is the “potential over-sensing of T wave”, original and filtered heart electric signals may be stored for professional (such as doctors) to view, or sent to professional and/or the subject himself and/or the subject's family members through text message, social media (such as WeChat) and so on. When first ratio value is greater than 10% and less than 25%, the first region label information is determined to be the focus label information. When the first ratio value is greater than or equal to 25%, the first region label information is determined to be the maintaining label information.

According to the heart signal monitoring method provided by the embodiment of the present disclosure, a purpose of determining, based on the peak information of the first monitoring wave corresponding to each of the plurality of first periods and the first threshold value information, first region label information of the first period is realized by performing calculation to obtain first difference value information according to the peak information of the first monitoring wave corresponding to each of the plurality of first periods and the first threshold value information; performing calculation to obtain first ratio information according to the first difference value information and the first threshold value information; and determining, based on the first ratio information, the first region label information. Accuracy of the determined first region label information may be improved by the embodiment of the present disclosure.

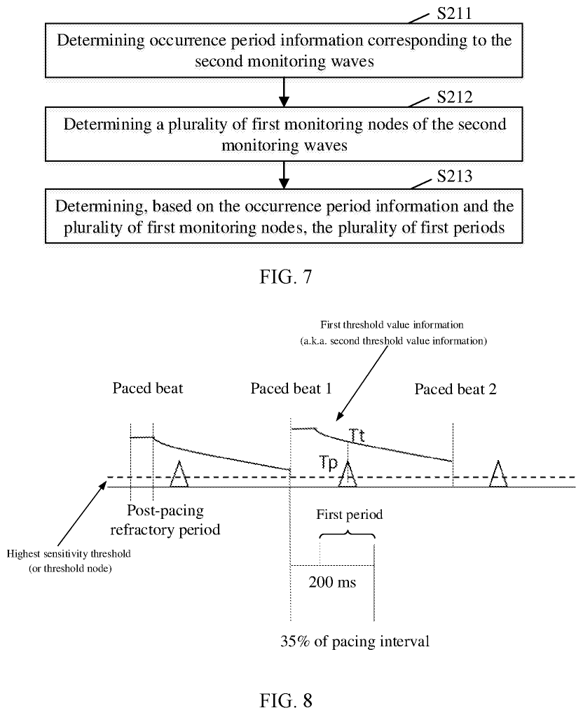

FIG. 7 is a schematic flowchart of determining, based on second monitoring waves corresponding to first monitoring waves, a plurality of first periods corresponding to the first monitoring waves according to an embodiment of the present disclosure. The embodiment shown in FIG. 7 is an extension on the basis of the embodiment shown in FIG. 4. A difference between the embodiments shown in FIG. 7 and FIG. 4 will be described in the following, and similarities will not be repeated herein.

As shown in FIG. 7, in an embodiment of the present disclosure, the determining, based on second monitoring waves corresponding to a first monitoring waves, a plurality of first periods corresponding to the first monitoring waves according to an embodiment of the present disclosure includes the following steps.

Step S211: determining occurrence period information corresponding to the second monitoring waves.

Exemplarily, the first monitoring wave is a T wave, and the second monitoring wave is an R wave. Accordingly, the occurrence period information corresponding to the second monitoring wave is determined based on peak values of adjacent the R waves, that is, time period information between peak values of the adjacent R waves (which may be represented by R-R) is determined to be the occurrence period information.

Step S212: determining a plurality of first monitoring nodes of the second monitoring waves.

For example, the second monitoring wave is the R wave, and the first monitoring node is a starting node at which the R wave is just to be sensed. Due to periodicity of the R wave, there will be a plurality of first monitoring nodes corresponding to a plurality of “consecutive” R waves.

Step S213: determining, based on the occurrence period information and the plurality of first monitoring nodes, the plurality of first periods.

Exemplarily, the plurality of first periods and the plurality of first monitoring nodes have a one-to-one correspondence. Specifically, for each of the plurality of first monitoring nodes mentioned in step S213, a node after a preset period (such as 200 ms or 100 ms, which is programmable) starting from each of the plurality of first monitoring nodes is determined as a start time node of the first period corresponding to the first monitoring node. Then, an end time node of the first period corresponding to the first monitoring node is determined based on a preset percentage of the occurrence period information (such as 35% time length of the occurrence period, that is 35% time length of an RR interval). Finally, the first period corresponding to the first monitoring node is determined based on the start time node and the end time node.

According to the heart signal monitoring method provided by the embodiment of the present disclosure, a purpose of determining, based on second monitoring waves in the electrocardiogram corresponding to the first monitoring waves, a plurality of first periods corresponding to the first monitoring waves is realized by determining occurrence period information corresponding to the second monitoring waves, determining a plurality of first monitoring nodes of the second monitoring waves, and determining, based on the occurrence period information and the plurality of first monitoring nodes, the plurality of first periods. The determination method of the first period in this embodiment fully takes into account the physiological connection between the first monitoring wave and second monitoring wave which have a related relationship with each other, and identifies a correlation between the first threshold value information and the first monitoring wave, so that the heart signals may be monitored with a higher accuracy within the preset monitoring time interval.

FIG. 8 is a schematic diagram of a waveform during a paced rhythm monitoring time interval according to an embodiment of the present disclosure. Specifically, FIG. 8 is a schematic diagram of a waveform of a T wave sensed within a paced rhythm monitoring time interval. FIG. 8 shows an example of the first threshold value information and so on. Those skilled in the art may clearly understand that FIG. 8 is only an example and does not constitute a sole limitation of the technical solution of the present disclosure.

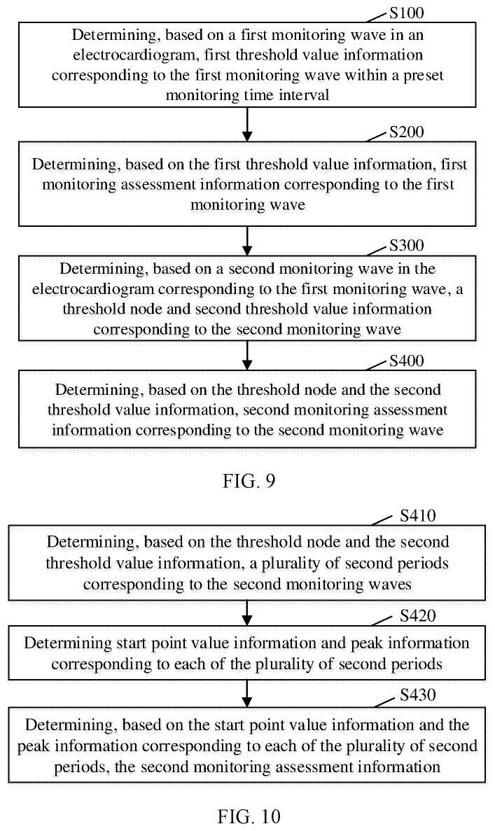

FIG. 9 is another schematic flowchart of a heart signal monitoring method according to an embodiment of the present disclosure. The embodiment shown in FIG. 9 is an extension on the basis of the embodiment shown in FIG. 3. A difference between the embodiments shown in FIG. 9 and FIG. 3 will be described in the following, and similarities will not be repeated herein.

Specifically, in an embodiment of the present disclosure, the preset monitoring time interval includes an intrinsic rhythm monitoring time interval. As shown in FIG. 9, the heart signal monitoring method provided by this embodiment further includes the following steps.

Step S300: determining, based on a second monitoring wave in the electrocardiogram corresponding to the first monitoring wave, a threshold node and second threshold value information corresponding to the second monitoring wave.

Exemplarily, the threshold node and the second threshold value information are used for sensing the second monitoring wave, and the second threshold value information is used for avoiding sensing the first monitoring wave. Optionally, the threshold node is used for filtering out wave ranges that have less monitoring significance (such as not meeting the preset monitoring conditions) in the second monitoring wave. In an embodiment of the present disclosure, the threshold node includes a threshold point and a time point, that is, an intersection point between the second threshold value information and the second monitoring wave. Exemplarily, the second threshold value information is varied, based on the second monitoring wave, with a certain regularity to avoid sensing the first monitoring wave. For example, the second threshold value information is linear or exponential decay information, so that sensing of the first monitoring wave may be avoided based on the linear or exponential decay threshold value information.

It should be noted that in some embodiments, the second threshold value information mentioned herein is the same as the first threshold value information mentioned in the above embodiments. In addition, in some other embodiments, the threshold node and the second threshold value information mentioned herein may be collectively referred to as threshold value information. For example, the threshold node may be a part of the second threshold value information. It should be understood that as long as the monitoring work mentioned in this embodiment may be completed in effect with the help of corresponding threshold value information, the division and designation of the threshold may be determined by those skilled in the art based on actual situations.

Step S400: determining, based on the threshold node and the second threshold value information, second monitoring assessment information corresponding to the second monitoring wave.

In an embodiment of the present disclosure, a second monitoring wave of one period is sensed based on the threshold node. Once the second monitoring wave is sensed, at the peak of the second monitoring wave corresponding to the sensed point, a sensing threshold value of a medical device is defined to decay along the second threshold value information over time until it decays to a next threshold node, and then, a second monitoring wave of the next period is sensed, thus achieving cyclic monitoring.

According to the heart signal monitoring method provided by the embodiment of the present disclosure, a purpose of simultaneously monitoring the first monitoring wave and the second monitoring wave within the intrinsic rhythm monitoring time interval is realized, thereby improving the accuracy and rationality of the heart signal monitoring.

It should be noted that step S300 and step S400 mentioned in the embodiment shown in FIG. 9 are not limited to being executed after step S200, but may also be executed before step S100, or be executed in parallel with step S100 and step S200, the embodiments of present disclosure do not specifically limit on this.

FIG. 10 is a schematic flowchart of determining, based on a threshold node and second threshold value information, second monitoring assessment information corresponding to a second monitoring wave according to an embodiment of the present disclosure. The embodiment shown in FIG. 10 is an extension on the basis of the embodiment shown in FIG. 9. A difference between the embodiments shown in FIG. 10 and FIG. 9 will be described in the following, and similarities will not be repeated herein.

As shown in FIG. 10, in an embodiment of the present disclosure, the determining, based on a threshold node and second threshold value information, second monitoring assessment information corresponding to a second monitoring wave includes the following steps.

Step S410: determining, based on the threshold node and the second threshold value information, a plurality of second periods corresponding to the second monitoring waves.

It should be understood that the second monitoring wave is periodic, and the plurality of second periods corresponding to the second monitoring waves may be determined based on the threshold nodes and the second threshold value information.

Exemplarily, time at which the second monitoring wave is sensed based on the threshold node may be taken as a start node of the second period, and an end node of the second period is determined based on a preset period.

Step S420: determining start point value information and peak information corresponding to each of the plurality of second periods.

Exemplarily, the peak information of the second monitoring wave corresponding to the second period refers to the peak information of the second monitoring wave sensed during this second period.

In an embodiment of the present disclosure, the method for determining the peak information of the second monitoring wave includes: taking the start node of the second period as a start point and determining the peak information of the second monitoring wave in a fixed period (such as 100 ms) along the time axis after the start point.

Step S430: determining, based on the start point value information and the peak information corresponding to each of the plurality of second periods, the second monitoring assessment information.

According to the heart signal monitoring method provided by the embodiment of the present disclosure, a purpose of determining, based on the threshold node and the second threshold value information, the second monitoring assessment information corresponding to the second monitoring wave is realized by determining, based on the threshold node and the second threshold value information, a plurality of second periods corresponding to the second monitoring waves; determining start point value information and peak information corresponding to each of the plurality of second periods; and determining, based on the start point value information and the peak information corresponding to each of the plurality of second periods, the second monitoring assessment information.

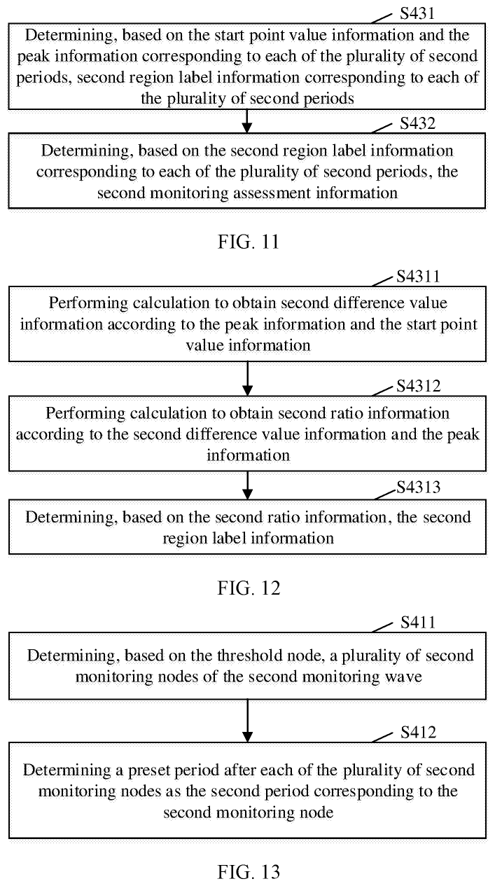

FIG. 11 is a schematic flowchart of determining, based on start point value information and peak information corresponding to each of a plurality of second periods, second monitoring assessment information according to an embodiment of the present disclosure. The embodiment shown in FIG. 11 is an extension on the basis of the embodiment shown in FIG. 10. A difference between the embodiments shown in FIG. 11 and FIG. 10 will be described in the following, and similarities will not be repeated herein.

As shown in FIG. 11, in an embodiment of the present disclosure, the determining, based on start point value information and peak information corresponding to each of a plurality of second periods, second monitoring assessment information includes the following steps.

Step S431: determining, based on the start point value information and the peak information corresponding to each of the plurality of second periods, second region label information corresponding to each of the plurality of second periods.

Exemplarily, the second region label includes an adjustment label, a focus label, and a maintaining label. Correspondingly, the second region label information includes adjustment label information, focus label information, and maintaining label information. The adjustment label represents that a parameter of a sensing amplifier (such as the threshold node and/or the second threshold value information mentioned above) corresponding to the heart signal needs to be adjusted. The focus label represents that the parameter of the sensing amplifier corresponding to the heart signal requires special attention but no adjustment temporarily. The maintain label represents that the sensing parameter corresponding to the heart signal is appropriate and do not need to be adjusted.

Step S432: determining, based on the second region label information corresponding to each of the plurality of second periods, the second monitoring assessment information.

Exemplarily, if the second region label information corresponding to each of six consecutive second periods mentioned in step S432 is the adjustment label information, then it indicates that the sensing parameter corresponding to the heart signal needs to be adjusted.

According to the heart signal monitoring method provided by the embodiment of the present disclosure. A purpose of determining the second monitoring assessment information on the basis of the start point value information and the peak information corresponding to each of the plurality of second periods is realized by determining, based on the start point value information and the peak information corresponding to each of the plurality of second periods, second region label information corresponding to each of the plurality of second periods, and determining, based on the second region label information corresponding to each of the plurality of second periods, the second monitoring assessment information. As mentioned before, heart signals of both patients with and without heart disease are very complex, therefore, rationality and accuracy of the determined second monitoring assessment information may be further improved by monitoring in different periods and determining the second monitoring assessment information in combination with the second region label information corresponding to each period.

FIG. 12 is a schematic flowchart of determining, based on start point value information and peak information corresponding to the second period, second region label information of second period according to an embodiment of the present disclosure. The embodiment shown in FIG. 12 is an extension on the basis of the embodiment shown in FIG. 11. A difference between the embodiments shown in FIG. 12 and FIG. 11 will be described in the following, and similarities will not be repeated herein.

As shown in FIG. 12, in an embodiment of the present disclosure, the determining, based on the start point value information and the peak information corresponding to the second period, second region label information of the second period includes the following steps.

Step S4311: performing calculation to obtain second difference value information according to the peak information and the start point value information.

Exemplarily, the second monitoring wave is an R wave, the peak information of the second monitoring wave is represented by Aₙ, and the start point value information of the second monitoring wave is represented by Bₙ. Then, the second difference value information R₁ₙ mentioned in step S4311 may be represented by the following equation (3).

R₁ₙ=Aₙ−Bₙ (3)

It should be understood that in this embodiment, n represents different second periods and is a positive integer greater than or equal to 1.

Step S4312: performing calculation to obtain second ratio information according to the second difference value information and the peak information.

Following the above notations, the second ratio information R₂ₙ may be represented by the following equation (4).

$${R}_{2 n} = \frac{{R}_{1 n}}{{A}_{n}} = \frac{{A}_{n} - {B}_{n}}{{A}_{n}} \qquad ( 4 )$$

Step S4313: determining, based on the second ratio information, the second region label information.

Specifically, when the second ratio information is less than a third preset value, the second region label information is determined to be the adjustment label information. And the third preset value is greater than or equal to 5%, and less than or equal to 15%. When the second ratio information is greater than or equal to the third preset value and less than a fourth preset value, the second region label information is determined to be the focus label information. Preferably, the fourth preset value is greater than or equal to 15%, and less than or equal to 30%. When the second ratio information is greater than or equal to the fourth preset value, the second region label information is determined to be the maintaining label information.

For example, if the second monitoring wave is the R wave and the second ratio information R₂ₙ is less than 10%, the second region label information is determined to be “potential under-sensing of R wave”, which is also known as the adjustment label information. Then, correspondingly, if the second region label information corresponding to each of six consecutive second periods is the “potential under-sensing of R wave”, a sensing amplifier parameter of the medical device may be changed (such as increasing a (preset) sensing gain level by one level, from 0.3 mV to 0.15 mV, or increasing the gain so that the second ratio information R₂ₙ may reach, for example, 25% or over 25%) and relevant information may be stored for doctors to refer to. If the second ratio information R₂ₙ is greater than 10% and less than 25%, the second region label information is determined to be “R wave sensed—requires attention”. Then, correspondingly, if the second region label information corresponding to each of six consecutive second periods is the “R wave sensed—requires attention”, which is also known as the focus label information, the relevant information may be stored and sent to the hardware device of relevant doctors for reference. When the second ratio information is greater than 25%, the second region label information is determined to be the maintaining label information.

According to the heart signal monitoring method provided by the embodiment of the present disclosure, a purpose of determining, based on the start point value information and the peak information corresponding to the second period, second region label information of the second period is realized by performing calculation to obtain second difference value information according to the peak information and the start point value information, performing calculation to obtain second ratio information according to the second difference value information and the peak information; and determining, based on the second ratio information, the second region label information. Accuracy of the determined second region label information may be improved by the embodiment of the present disclosure.

In an embodiment of the present disclosure, if Aₙ equals to a maximum output of the sensing amplifier, it means that the R wave has caused saturation of the sensing amplifier output, and the gain level of the sensing amplifier may be reduced, such as from 0.15 mv to 0.3 mv. Alternatively, the gain level of the sensing amplifier may be reduced until Aₙ continuously drops below the maximum sensing amplifier output. Alternatively, without making any changes, this information may be labeled to remind doctors to pay attention. No matter how it is processed, relevant information may also be stored for doctors to refer to.

FIG. 13 is a schematic flowchart of determining, based on a threshold node and second threshold value information, a plurality of second periods corresponding to second monitoring waves according to an embodiment of the present disclosure. The embodiment shown in FIG. 13 is an extension on the basis of the embodiment shown in FIG. 10. A difference between the embodiments shown in FIG. 13 and FIG. 10 will be described in the following, and similarities will not be repeated herein.

As shown in FIG. 13, in an embodiment of the present disclosure, the determining, based on a threshold node and second threshold value information, a plurality of second periods corresponding to second monitoring waves includes the following steps.

Step S411: determining, based on the threshold node, a plurality of second monitoring nodes of the second monitoring wave.

For example, as the second monitoring wave is periodic, the time node at which the threshold node is initially reached in each period is determined as the second monitoring node.

It should be noted that in some embodiments, the second monitoring node mentioned in this embodiment is the first monitoring node mentioned in the above embodiment. In other words, the node at which the R wave being sensed initially in each period is the node at which the R wave being sensed initially based on the threshold node.

Step S412: determining a preset period after each of the plurality of second monitoring nodes as the second period corresponding to the second monitoring node.

Exemplarily, the second monitoring node mentioned in this embodiment is the same monitoring node as the first monitoring node mentioned in the above-mentioned embodiments.

According to the heart signal monitoring method, a purpose of determining, based on the threshold node and second threshold value information, a plurality of second periods corresponding to the second monitoring waves is realized. The determination method of the second period mentioned in this embodiment achieves more accurate monitoring of heart signals. Especially when the first monitoring wave is a T wave and the second monitoring wave is an R wave, or the first monitoring wave is an FFR wave and the second monitoring wave is a P wave, this embodiment may fully consider an actual correlation between the first monitoring wave and the second monitoring wave which have a related relationship with each other, so that the accuracy of the heart signal monitored may be improved within the intrinsic rhythm monitoring time interval.

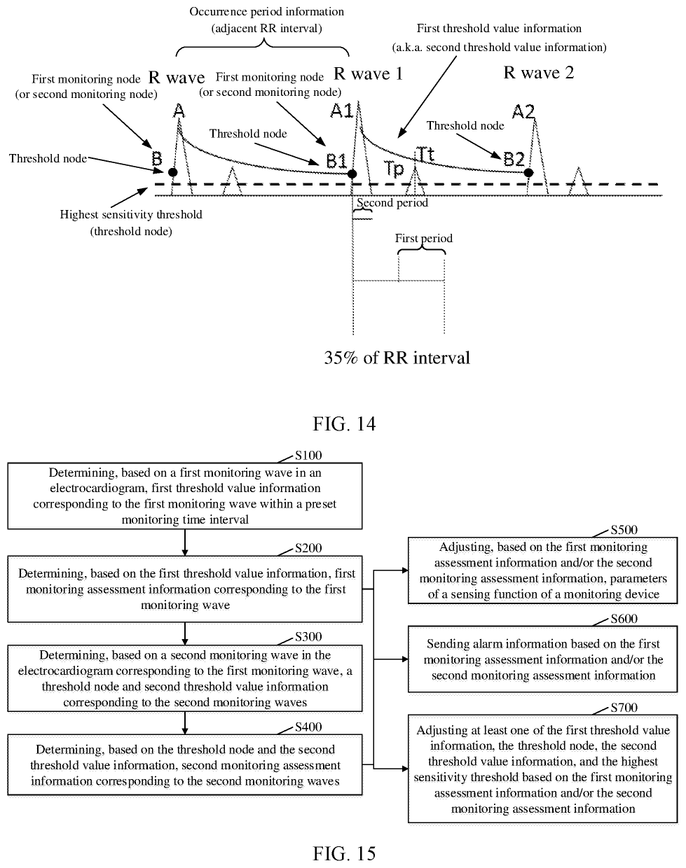

FIG. 14 is a schematic diagram of a waveform during a self-rhythm monitoring time interval according to an embodiment of the present disclosure. Specifically, FIG. 14 is a schematic diagram of a waveform of an R wave and a T wave sensed during an intrinsic rhythm monitoring time interval, which may be used to characterize a ventricular condition of the subject. Similar to FIG. 8, FIG. 14 provides examples of first threshold value information, first monitoring node, and so on. Therein, the threshold node may be a cutoff node for the second threshold value information. In different second periods, specific values of the threshold nodes may be different, that is, the values of B1 and B2 may be different.

In an embodiment of the present disclosure, the heart signal monitoring method in the embodiments mentioned above may be performed at a specific time on a natural day. For example, the heart signal monitoring method may be performed twice a natural day, once at night (such as starting at 3 am), and once during the day (such as starting at 3 pm). For example, the heart signal monitoring method may also be performed four times a natural day, that is, performed every six hours, at 3 am, 9 am, 3 μm, and 9 pm respectively. The monitoring may also be performed every 15 minutes or every hour. The embodiments of the present disclosure do not specifically limit on this.

FIG. 15 is still another schematic flowchart of a heart signal monitoring method according to an embodiment of the present disclosure. The embodiment shown in FIG. 15 is an extension on the basis of the embodiment shown in FIG. 9. A difference between the embodiments shown in FIG. 15 and FIG. 9 will be described in the following, and similarities will not be repeated herein.

As shown in FIG. 15, the heart signal monitoring method provided by an embodiment of the present disclosure further includes the following steps.

Step S500: adjusting, based on the first monitoring assessment information and/or the second monitoring assessment information, parameters of a sensing function of a monitoring device.

Exemplarily, when the first monitoring assessment information is determined to be that the first monitoring wave is nearly to be sensed, and/or the second monitoring assessment information is determined to be that the second monitoring wave is nearly not to be sensed, parameters of a sensing function of a monitoring device are adjusted based on the first monitoring assessment information and/or the second monitoring assessment information. Alternatively, when the first region label information is the adjustment label information or the focus label information, and/or when the second region label information is the adjustment label information or the focus label information, parameters of the sensing function of the monitoring device are adjusted based on the first monitoring assessment information and/or the second monitoring assessment information.

Step S600: sending alarm information based on the first monitoring assessment information and/or the second monitoring assessment information.

Exemplarily, when the first monitoring assessment information is determined to be that the first monitoring wave is nearly to be sensed, and/or the second monitoring assessment information is determined to be that the second monitoring wave is nearly not to be sensed, alarm information may be sent to the communication devices (such as mobile phones, tablet computers, auxiliary communication equipment specialized for medical equipment, and so on) of doctors and/or the subject's family members and/or the subject based on the first monitoring assessment information and/or the second monitoring assessment information. Alternatively, when the first region label information is the adjustment label information or the focus label information, and/or when the second region label information is the adjustment label information or the focus label information, alarm information may be sent to the communication devices (such as mobile phones, tablet computers, auxiliary communication equipment specialized for medical equipment, and so on) of doctors and/or the subject's family members and/or the subject based on the first monitoring assessment information and/or the second monitoring assessment information.

In some embodiments, the alarm information may be divided into different levels based on severity of condition in patients. For example, the first level is to immediately trigger an alarm and immediately call emergency numbers such as 110 and/or 120; the second level is to temporarily suspend the alarm but record the warning instance in the form of numbers or charts and notify doctors to follow this information online when they have time; alternatively, the third level is to provide recorded information to doctor for reference when the patients go to the hospital next time.

Step S700: adjusting, based on the first monitoring assessment information and/or the second monitoring assessment information, at least one of the first threshold value information, the threshold node, the second threshold value information, and a highest sensitivity threshold information.

Exemplarily, when the first monitoring assessment information is determined to be that the first monitoring wave is nearly to be sensed, and/or the second monitoring assessment information is determined to be that the second monitoring wave is nearly not to be sensed, the sensing parameter of the sensing amplifier is adjusted based on the first monitoring assessment information and/or the second monitoring assessment information, thereby adjusting at least one of the first threshold value information, the threshold node, the second threshold value information, and the highest sensitivity threshold. Alternatively, when the first region label information is the adjustment label information or the focus label information, and/or when the second region label information is the adjustment label information or the focus label information, the sensing parameter of the sensing amplifier is adjusted based on the first monitoring assessment information and/or the second monitoring assessment information, thereby adjusting at least one of the first threshold value information, the threshold node, the second threshold value information, and the highest sensitivity threshold.

Exemplarily, when the first monitoring assessment information is determined to be that the first monitoring wave is nearly to be sensed, and/or the second monitoring assessment information is determined to be that the second monitoring wave is nearly not to be sensed, this method further includes: storing relevant data corresponding to the first monitoring assessment information and/or the second monitoring assessment information for information post-processing and evaluating by a doctor, where the relevant data includes the first monitoring wave, the second monitoring wave, as well as the first threshold value information, the second threshold value information, and so on.

It should be noted that Step S500, Step S600 and Step S700 can also be executed after Step S200, the embodiments of present disclosure do not specifically limit on this. According to the embodiments of present disclosure, those skilled in the art may understand that parameter adjustment, alarm prompt, and data storage may be performed based on the first monitoring assessment information and the second monitoring assessment information respectively, as well as parameter adjustment, alarm prompt, and data storage may be performed based on both the first monitoring assessment information and the second monitoring assessment information.

The embodiments of the present disclosure may provide monitoring equipment with appropriate sensing sensitivity thresholds for the subject based on their actual physical condition, and may timely detect potential risks and send them to concerned person in the form of alarm information, so that the subject may be treated more promptly.

In another embodiment of the present disclosure, before steps S500, S600, and S700 being performed, verification needs to be performed based on the first monitoring assessment information and/or the second monitoring assessment information.

On the other hand, an embodiment of present disclosure provides a heart signal monitoring method, which includes: determining a threshold node and second threshold value information corresponding to a second monitoring wave based on the second monitoring wave in an electrocardiogram corresponding to a first monitoring wave within a preset monitoring time interval, where the second threshold value information and the threshold node are used for sensing the second monitoring wave, and the second threshold value information is used for avoiding sensing the first monitoring wave; and determining second monitoring assessment information corresponding to the second monitoring wave based on the threshold node and the second threshold value information.

### Exemplary Apparatus

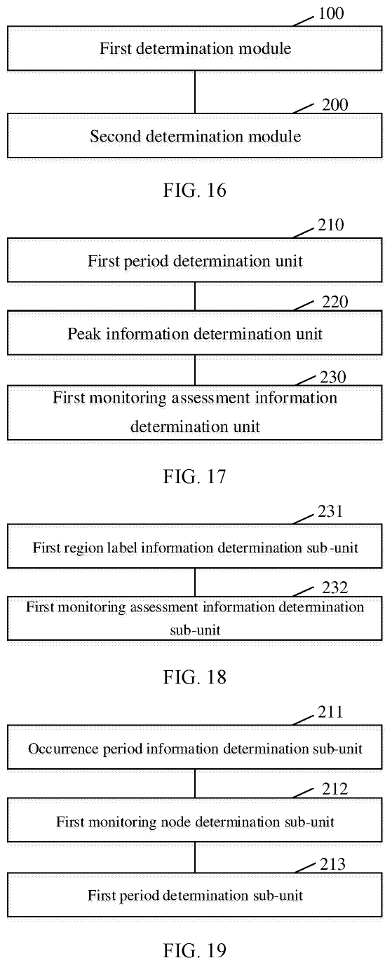

FIG. 16 is a schematic structural diagram of a heart signal monitoring apparatus according to an embodiment of the present disclosure. As shown in FIG. 16, the heart signal monitoring apparatus provided by this embodiment includes:
a first determination module 100, configured to determine, based on a first monitoring wave in an electrocardiogram, first threshold value information corresponding to the first monitoring wave within a preset monitoring time interval; and
a second determination module 200, configured to determine, based on the first threshold value information, first monitoring assessment information corresponding to the first monitoring wave.

FIG. 17 is a schematic structural diagram of a second determination module according to an embodiment of the present disclosure. The embodiment shown in FIG. 17 is an extension on the basis of the embodiment shown in FIG. 16. A difference between the embodiments shown in FIG. 17 and FIG. 16 will be described in the following, and similarities will not be repeated herein.

As shown in FIG. 17, in an embodiment of the present disclosure, the second determination module 200 includes:
a first period determination unit 210, configured to determine, based on second monitoring waves in the electrocardiogram corresponding to the first monitoring wave, a plurality of first periods corresponding to the first monitoring wave;
a peak information determination unit 220, configured to determine peak information of the first monitoring wave corresponding to each of the plurality of first periods; and
a first monitoring assessment information determination unit 230, configured to determine, based on the peak information of the first monitoring wave corresponding to each of the plurality of first periods and the first threshold value information, the first monitoring assessment information.

FIG. 18 is a schematic structural diagram of a first monitoring assessment information determination unit according to an embodiment of the present disclosure. The embodiment shown in FIG. 18 is an extension on the basis of the embodiment shown in FIG. 17. A difference between the embodiments shown in FIG. 18 and FIG. 17 will be described in the following, and similarities will not be repeated herein.

As shown in FIG. 18, in an embodiment of the present disclosure, the first monitoring assessment information determination unit 230 includes:
a first region label information determination sub-unit 231, configured to determine, based on the peak information of the first monitoring wave corresponding to each of the plurality of first periods and the first threshold value information, first region label information corresponding to each of the plurality of first periods; and
a first monitoring assessment information determination sub-unit 232, configured to determine, based on the first region label information corresponding to each of the plurality of first periods, the first monitoring assessment information.

FIG. 19 is a schematic structural diagram of a first period determination unit according to an embodiment of the present disclosure. The embodiment shown in FIG. 19 is an extension on the basis of the embodiment shown in FIG. 17. A difference between the embodiments shown in FIG. 19 and FIG. 17 will be described in the following, and similarities will not be repeated herein.

As shown in FIG. 19, in an embodiment of the present disclosure, the first period determination unit 210 includes:
an occurrence period information determination sub-unit 211, configured to determine occurrence period information corresponding to the second monitoring waves;
a first monitoring node determination sub-unit 212, configured to determine a plurality of first monitoring nodes of the second monitoring wave; and
a first period determination sub-unit 212, configured to determine, based on the occurrence period information and the plurality of first monitoring nodes, the plurality of first periods.

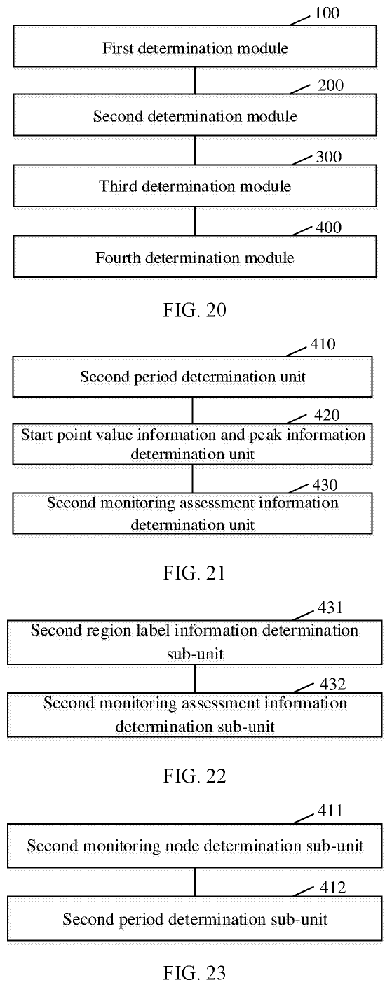

FIG. 20 is another schematic structural diagram of a heart signal monitoring apparatus according to an embodiment of the present disclosure. The embodiment shown in FIG. 20 is an extension on the basis of the embodiment shown in FIG. 16. A difference between the embodiments shown in FIG. 20 and FIG. 16 will be described in the following, and similarities will not be repeated herein.

As shown in FIG. 20, the heart signal monitoring apparatus provided by this embodiment further includes:
a third determination module 300, configured to determine, based on a second monitoring wave in an electrocardiogram corresponding to a first monitoring wave, a threshold node and second threshold value information corresponding to the second monitoring wave; and
a fourth determination module 400, configured to determine, based on the threshold node and the second threshold value information, second monitoring assessment information corresponding to the second monitoring wave.

FIG. 21 is a schematic structural diagram of a fourth determination module according to an embodiment of the present disclosure. The embodiment shown in FIG. 21 is an extension on the basis of the embodiment shown in FIG. 20. A difference between the embodiments shown in FIG. 21 and FIG. 20 will be described in the following, and similarities will not be repeated herein.

As shown in FIG. 21, in an embodiment of the present disclosure, the fourth determination module 400 includes:
a second period determination unit 410, configured to determine, based on the threshold node and the second threshold value information, a plurality of second periods corresponding to the second monitoring waves;
a start point value information and peak information determination unit 420, configured to determine start point value information and peak information corresponding to each of the plurality of second periods; and
a second monitoring assessment information determination unit 430, configured to determine, based on the start point value information and the peak information corresponding to each of the plurality of second periods, the second monitoring assessment information.

FIG. 22 is a schematic structural diagram of a second monitoring assessment information determination unit according to an embodiment of the present disclosure. The embodiment shown in FIG. 22 is an extension on the basis of the embodiment shown in FIG. 21. A difference between the embodiments shown in FIG. 22 and FIG. 21 will be described in the following, and similarities will not be repeated herein.

As shown in FIG. 22, in an embodiment of the present disclosure, the second monitoring assessment information determination unit 430 includes:
a second region label information determination sub-unit 431, configured to determine, based on the start point value information and the peak information corresponding to each of the plurality of second periods, second region label information corresponding to each of the plurality of second periods; and
a second monitoring assessment information determination sub-unit 432, configured to determine, based on the second region label information corresponding to each of the plurality of second periods, the second monitoring assessment information.

FIG. 23 is a schematic structural diagram of a second period determination unit according to an embodiment of the present disclosure. The embodiment shown in FIG. 23 is an extension on the basis of the embodiment shown in FIG. 21. A difference between the embodiments shown in FIG. 23 and FIG. 21 will be described in the following, and similarities will not be repeated herein.

As shown in FIG. 23, in an embodiment of the present disclosure, the second period determination unit 410 includes:
a second monitoring node determination sub-unit 411, configured to determine, based on the threshold node, a plurality of second monitoring nodes of the second monitoring wave; and
a second period determination sub-unit 412, configured to determine a preset period after each of the plurality of second monitoring nodes as the second period corresponding to the second monitoring node.

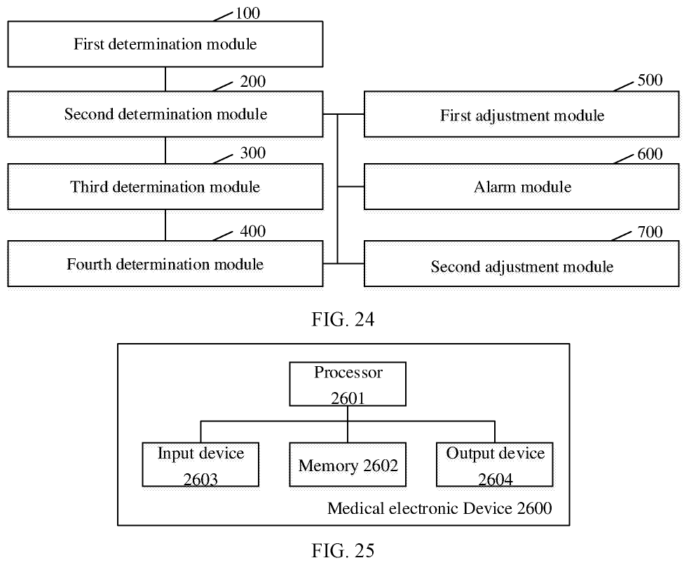

FIG. 24 is still another schematic structural diagram of a heart signal monitoring apparatus according to an embodiment of the present disclosure. The embodiment shown in FIG. 24 is an extension on the basis of the embodiment shown in FIG. 20. A difference between the embodiments shown in FIG. 24 and FIG. 20 will be described in the following, and similarities will not be repeated herein.

As shown in FIG. 24, the heart signal monitoring apparatus provided by this embodiment further includes:
a first adjustment module 500, configured to adjust, based on the first monitoring assessment information and/or the second monitoring assessment information, parameters of a sensing function of a monitoring device;
an alarm module 600, configured to send alarm information based on the first monitoring assessment information and/or the second monitoring assessment information; and
a second adjustment module 700, configured to adjust, based on the first monitoring assessment information and/or the second monitoring assessment information, at least one of the first threshold value information, the threshold node, the second threshold value information, and a highest sensitivity threshold.

Exemplarily, the second adjustment module 700 is also configured to store relevant data corresponding to the first monitoring assessment information and/or the second monitoring assessment information for information post-processing and evaluating by a doctor. Therein. the relevant data includes the first monitoring wave, the second monitoring wave, as well as the first threshold value information, the second threshold value information, and so on.

On the other hand, an embodiment of present disclosure provides a heart signal monitoring apparatus, which includes: a third determination module, configured to determine, based on the second monitoring wave in an electrocardiogram corresponding to a first monitoring wave, a threshold node and second threshold value information corresponding to the second monitoring wave, where the second threshold value information and the threshold node are used for sensing the second monitoring wave, and the second threshold value information is used for avoiding sensing of the first monitoring wave; and a fourth determination module, configured to determine, based on the threshold node and the second threshold value information, second monitoring assessment information corresponding to the second monitoring wave.

It should be understood that the operation and functions of the modules, units, and sub-units included in the heart signal monitoring apparatus provided in FIG. 16 to FIG. 24 may refer to the heart signal monitoring methods provided in FIG. 3 to FIG. 13 above. In order to avoid repetition, similarities will not be repeated herein.

In addition, it should be noted that the heart signal monitoring apparatus mentioned in the above embodiment can have its own heart signal monitoring method or can be combined with existing medical equipment/devices to achieve the heart signal monitoring method mentioned in the above embodiment by the reference parameters collected by the data collection function of the existing medical equipment/devices, thereby achieving monitoring heart arrhythmia events (including but not limited to premature ventricular complex (PVC) or premature atrial complex (PAC), non-persistent ventricular tachycardia (VT), atrial fibrillation (AF), ventricular tachycardia (VT), and ventricular fibrillation (VF)) through the heart signal monitoring method.

A medical electronic device according to an embodiment of the present disclosure will be described below with reference to FIG. 25. FIG. 25 is a schematic structural diagram of an electronic device according to an embodiment of the present disclosure.

As shown in FIG. 25, the medical electronic device 2600 includes one or more processors 2601 and memory 2602.

The processor 2601 can be a central processing unit (CPU) or other form of processing unit with data processing and/or instruction execution capabilities, and can control other components in the medical electronic device 2600 to perform desired functions.

The memory 2602 may include one or more computer program products, which may include various forms of computer-readable storage media, such as volatile memory and/or non volatile memory. The volatile memory may include, for example, random access memory (RAM) and/or cache memory. The non-volatile memory may include, for example, read-only memory (ROM), hard disk, flash memory, and so on. One or more computer program instructions can be stored on the computer-readable storage medium, and the processor 2601 can run the program instructions to achieve the heart signal monitoring methods and/or other desired functions of the various embodiments of the present disclosure as described above. And the computer-readable storage medium may further store various contents such as heart rate information.

In an example, the electronic device 2600 may also include an input device 2603 and an output device 2604. These components are interconnected by a bus system and/or other forms of connection mechanism (not shown).

The input device 2603 may also include, for example, a keyboard, a mouse, and so on.

The output device 2604 may output various kinds of information, such as first monitoring assessment information, to the outside. The output device 2604 may include, for example, a display, a speaker, a printer, a communication network and remote output device connected on it, and so on.

To simplify, only some of the components related to the present disclosure in the electronic device 2600 are shown in FIG. 25 and components such as bus, input/output interface and so on are omitted. Moreover, the electronic device 2600 may also include any other appropriate components according to the specific disclosure.

Beyond the above methods and devices, the embodiments of the present disclosure may also be computer program products, such as computer program instructions, which enable the processor to perform the steps of the data processing method as described in any one of above embodiments when run by the processor.

The computer program product may write program code for executing the operation of the embodiments of the present disclosure in any combination of one or more programming languages. The programming languages include object-oriented programming languages, such as Java, C++, and also include conventional procedural programming languages, such as “C” language or similar programming languages. The program code may be completely executed on the user's computing device, partially executed on the user's device, executed as an independent software package, partially executed on the user's computing device and partially executed on the remote computing device, or completely executed on the remote computing device or processor.

In addition, the embodiment of the present disclosure may also be computer-readable storage media, on which computer executable instructions are stored. When the executable instructions are executed by a processor, the heart signal monitoring described above is implemented.

The computer-readable storage medium may adopt any combination of one or more readable media. The readable medium can be a readable signal medium or a readable storage medium. A readable storage medium may include, but is not limited to, systems, devices or devices of electricity, magnetism, light, electromagnetism, infrared, semiconductors, or any combination of the above. More specific examples of readable storage media (non-exhaustive list) include: electrical connections with one or more wires, portable disks, hard disks, random access memory (RAM), read-only memory (ROM), erasable programmable read-only memory (EPROM or flash memory), optical fibers, portable compact disk read-only memory (CD-ROM), optical storage devices, magnetic storage devices, or any suitable combination of the above.

The above describes the basic principle of the present disclosure with reference to specific embodiments. However, it should be noted that the advantages, superiorities, effects and so on mentioned in the present disclosure are only examples, but not limitations. It cannot be considered that these advantages, superiorities, effects and so on are necessary for each embodiment of the present disclosure. In addition, the specific details disclosed above are only for the purpose of example and easy understanding, but not for limitation. The present disclosure is not limited to the above specific details.

The block diagrams of means, apparatuses, devices and systems involved in the present disclosure are only illustrative examples and are not intended to require or imply that they must be connected, disposed and configured in the manner shown in the block diagram. As those skilled in the art will recognize, these means, apparatuses, devices and systems can be connected, disposed and configured in any way. The terms such as “include”, “contain”, “have” and so on are open-class words, and referring to “include but not limited to”, and can be used interchangeably. The terms “or” and “and” refer to the terms “and/or” and can be used interchangeably, unless the context clearly indicate otherwise. The term “such as” refers to the terms “such as but not limited to” and can be used interchangeably.

It should also be noted that each component or step in the apparatus, device and method of the present disclosure can be decomposed and/or reassembled. The decomposition and/or recombination shall be considered as the equivalent of the present disclosure.

The above description of the disclosed aspects is provided to enable any person skilled in the art to make or use the present disclosure. Any modification to these aspects is obvious to a person skilled in the art, and the general principles defined herein can be applied to other aspects without departing from the protection scope of the present disclosure. Therefore, the present disclosure is not intended to be limited to the aspects shown herein, but to the widest range consistent with the principles and novel features disclosed herein.

The above description has been provided for the purpose of illustration and description. Furthermore, this description is not intended to limit the protection scope of the present disclosure to the form disclosed herein. Although several exemplary aspects and embodiments have been discussed above, those skilled in the art will recognize certain variations, modifications, changes, additions, and sub-combinations thereof.

What is claimed is:

1. A heart signal monitoring method, comprising:
determining, based on a first monitoring wave in an electrocardiogram, first threshold value information corresponding to the first monitoring wave within a preset monitoring time interval, wherein the first threshold value information is used for monitoring the first monitoring wave; and
determining, based on the first threshold value information, first monitoring assessment information corresponding to the first monitoring wave, wherein the first monitoring assessment information comprises whether the first monitoring wave being nearly to be sensed.

2. The heart signal monitoring method according to claim 1, wherein the determining, based on the first threshold value information, first monitoring assessment information corresponding to the first monitoring wave comprises:
determining, based on second monitoring waves in the electrocardiogram corresponding to the first monitoring wave, a plurality of first periods corresponding to the first monitoring wave;
determining peak information of the first monitoring wave corresponding to each of the plurality of first periods; and
determining, based on the peak information of the first monitoring wave corresponding to each of the plurality of first periods and the first threshold value information, the first monitoring assessment information;
wherein the first monitoring wave is a T wave, and the second monitoring wave is an R wave; and/or the first monitoring wave is a far-field R (FFR) wave, and the second monitoring wave is a P wave.

3. The heart signal monitoring method according to claim 2, wherein the determining, based on the peak information of the first monitoring wave corresponding to each of the plurality of first periods and the first threshold value information, the first monitoring assessment information comprises:
determining, based on the peak information of the first monitoring wave corresponding to each of the plurality of first periods and the first threshold value information, first region label information corresponding to each of the plurality of first periods, wherein the first region label information comprises adjustment label information, focus label information, and maintaining label information; and
determining, based on the first region label information corresponding to each of the plurality of first periods, the first monitoring assessment information.

4. The heart signal monitoring method according to claim 3, wherein the determining, based on the peak information of the first monitoring wave corresponding to each of the plurality of first periods and the first threshold value information, first region label information of the first period corresponding to each of the plurality of first periods comprises:
performing calculation to obtain first difference value information according to the peak information of the first monitoring wave corresponding to each of the plurality of first periods and the first threshold value information;
performing calculation to obtain first ratio information according to the first difference value information and the first threshold value information; and
determining, based on the first ratio information, the first region label information.

5. The heart signal monitoring method according to claim 4, wherein the determining, based on the first ratio information, the first region label information comprises:
when the first ratio information is less than a first preset value, determining the first region label information to be the adjustment label information, wherein the first preset value is greater than or equal to 5%, and less than or equal to 15%; and/or
when the first ratio information is greater than or equal to the first preset value and less than a second preset value, determining the first region label information to be the focus label information, wherein the second preset value is greater than or equal to 15%, and less than or equal to 30%;
wherein the determining, based on the first region label information corresponding to each of the plurality of first periods, the first monitoring assessment information comprises:
when the first region label information corresponding to each of a plurality of consecutive first periods is the adjustment label information, determining that the first monitoring wave is nearly to be sensed, wherein the plurality of consecutive first periods comprise two or more than two first periods.

6. The heart signal monitoring method according to claim 2, wherein the determining, based on second monitoring waves in the electrocardiogram corresponding to the first monitoring wave, a plurality of first periods corresponding to the first monitoring wave comprises:
determining occurrence period information corresponding to the second monitoring waves;
determining a plurality of first monitoring nodes of the second monitoring waves;
determining a node after a preset period starting from each of the plurality of first monitoring nodes as a start time node of the first period corresponding to the first monitoring node, wherein the plurality of first periods and the plurality of first monitoring nodes have a one-to-one correspondence;
determining, based on a preset percentage of the occurrence period information, an end time node of the first period corresponding to the first monitoring node; and
determining, based on the start time node and the end time node, the first period corresponding to the first monitoring node.

7. A heart signal monitoring method, comprising:
determining, based on a second monitoring wave in an electrocardiogram corresponding to a first monitoring wave, a threshold node and second threshold value information corresponding to the second monitoring wave within a preset monitoring time interval, wherein the threshold node and the second threshold value information are used for sensing the second monitoring wave, and the second threshold value information is used for avoiding sensing the first monitoring wave; and
determining, based on the threshold node and the second threshold value information, second monitoring assessment information corresponding to the second monitoring wave, wherein the second monitoring assessment information comprises whether the second monitoring wave being nearly not to be sensed.

8. The heart signal monitoring method according to claim 7, wherein the determining, based on the threshold node and the second threshold value information, second monitoring assessment information corresponding to the second monitoring wave comprises:
determining, based on the threshold node and the second threshold value information, a plurality of second periods corresponding to the second monitoring wave, wherein the second threshold value information is linear or exponential decay information;
determining start point value information and peak information corresponding to each of the plurality of second periods; and
determining, based on the start point value information and the peak information corresponding to each of the plurality of second periods, the second monitoring assessment information.

9. The heart signal monitoring method according to claim 8, wherein the determining, based on the start point value information and the peak information corresponding to each of the plurality of second periods, the second monitoring assessment information comprises:
determining, based on the start point value information and the peak information corresponding to each of the plurality of second periods, second region label information corresponding to each of the plurality of second periods, wherein the second region label information comprises adjustment label information, focus label information, and maintaining label information; and
determining, based on the second region label information corresponding to each of the plurality of second periods, the second monitoring assessment information.

10. The heart signal monitoring method according to claim 9, wherein the determining, based on the start point value information and the peak information corresponding to each of the plurality of second periods, second region label information corresponding to each of the plurality of second periods comprises:
performing calculation to obtain second difference value information according to the peak information and the start point value information;
performing calculation to obtain second ratio information according to the second difference value information and the peak information; and
determining, based on the second ratio information, the second region label information.

11. The heart signal monitoring method according to claim 10, wherein the determining, based on the second ratio information, the second region label information comprises:
when the second ratio information is less than a third preset value, determining the second region label information to be the adjustment label information, wherein the third preset value is greater than or equal to 5%, and less than or equal to 15%; and/or
when the second ratio information is greater than or equal to the third preset value and less than a fourth preset value, determining the second region label information to be the focus label information, wherein the fourth preset value is greater than or equal to 15%, and less than or equal to 30%;
wherein the determining the second monitoring assessment information based on the second region label information corresponding to each of the plurality of second periods comprises:
when the second region label information corresponding to each of a plurality of consecutive first periods is the adjustment label information, determining that the second monitoring wave is nearly not to be sensed, wherein the plurality of consecutive second periods comprise two or more than two second periods.

12. The heart signal monitoring method according to claim 7, wherein the determining, based on the threshold node and the second threshold value information, a plurality of second periods corresponding to the second monitoring wave comprises:
determining, based on the threshold nodes, a plurality of second monitoring nodes of the second monitoring wave; and
determining a preset period after each of the plurality of second monitoring nodes as the second period corresponding to the second monitoring node.

13. The heart signal monitoring method according to claim 7, further comprising:
determining, based on the first monitoring wave in the electrocardiogram, first threshold value information corresponding to the first monitoring wave within the preset monitoring time interval, wherein the first threshold value information is used for monitoring the first monitoring wave; and
determining, based on the first threshold value information, first monitoring assessment information corresponding to the first monitoring wave, wherein the first monitoring assessment information comprises whether the first monitoring wave being nearly to be sensed.

14. The heart signal monitoring method according to claim 13, further comprising:
when the first monitoring assessment information is that the first monitoring wave is nearly to be sensed, and/or the second monitoring assessment information is that the second monitoring wave is nearly not to be sensed,
adjusting, based on the first monitoring assessment information and/or the second monitoring assessment information, parameters of a sensing function of a monitoring device; and/or
sending alarm information based on the first monitoring assessment information and/or the second monitoring assessment information; and/or
adjusting, based on the first monitoring assessment information and/or the second monitoring assessment information, at least one of the first threshold value information, the threshold node, the second threshold value information, and a highest sensitivity threshold; and/or
storing relevant data corresponding to the first monitoring assessment information and/or the second monitoring assessment information for information post-processing and evaluating by a doctor.

15. The heart signal monitoring method according to claim 14, wherein the monitoring device comprises an implantable medical device, and the implantable medical device comprises a pacemaker.

16. The heart signal monitoring method according to claim 14, wherein the adjusting, based on the first monitoring assessment information and/or the second monitoring assessment information, at least one of the first threshold value information, the threshold node, the second threshold value information, and a highest sensitivity threshold information comprises:
adjusting, based on the first monitoring assessment information and/or the second monitoring assessment information, a sensing parameter of a sensing amplifier, to adjust at least one of the first threshold value information, the threshold node, the second threshold value information, and the highest sensitivity threshold.

17. A non-transitory computer-readable storage medium, wherein a computer program is stored thereon for performing the heart signal monitoring method according to claim 1.

18. A medical electronic device, comprising:
a processor; and
a memory for storing executable instructions of the processor,
wherein the processor is used for implementing the heart signal monitoring method according to claim 1.

19. A non-transitory computer-readable storage medium, wherein a computer program is stored thereon for performing the heart signal monitoring method according to claim 7.

20. A medical electronic device, comprising:
a processor; and
a memory, configured to store executable instructions of the processor,
wherein the processor is used for implementing the heart signal monitoring method according to claim 7.
